(19) United States
(12) Patent Application Publication
(10) Pub. No.: US 2020/0176127 A1
(43) Pub. Date: Jun. 4, 2020

(54) SYSTEMS AND METHODS FOR GUIDELINE CONCORDANCE

(71) Applicant: Flatiron Health, Inc., New York, NY (US)

(72) Inventors: Jessie Tseng, New York, NY (US); James Tyler Martineau, New York, NY (US); Vivien Liu Ekuan, Los Altos, CA (US); Dominique Connolly, Jersey City, NJ (US); Neal J. Meropol, New York, NY (US); Robert Jeffrey Green, West Palm Beach, FL (US); Harvey James Hamrick, JR., Atlanta, GA (US); Alison Fugaro, Atlanta, GA (US); Helaina Talcott, New York, NY (US); Amila Meera Patel, Seattle, WA (US); Michael Tyler Haydell, New York, NY (US); Kyle Ritter, College Station, TX (US)

(73) Assignee: Flatiron Health, Inc., New York, NY (US)

(21) Appl. No.: 16/702,113

(22) Filed: Dec. 3, 2019

Related U.S. Application Data
(60) Provisional application No. 62/774,621, filed on Dec. 3, 2018.

(51) Int. Cl. G16H 70/20; G16H 70/40; G16H 10/60; G06F 16/338; G06F 3/048

(52) U.S. Cl. CPC G16H 70/20; G16H 70/40; G06F 3/048; G06F 16/338; G16H 10/60

(57) ABSTRACT

A system for providing guideline concordance may include at least one processing device programmed to receive, via a graphical user interface of a user device, a search term associated with a drug; access a structured database to identify, based on the search term, a description of at least one regimen that includes the search term; display, via the graphical user interface, a selectable identifier of the at least one regimen; receive, via the graphical user interface, a selection of a regimen, wherein the regimen is associated with the drug; generate, based on the structured database, one or more indications that are concordant for the regimen; receive, via the graphical user interface, a selection of a concordant indication; and store, in an electronic health record database, a patient record with information identifying the selected regimen and the selected indication.

### CROSS REFERENCE TO RELATED APPLICATION

This application claims the benefit of priority of U.S. Provisional Application No. 62/774,621, filed on Dec. 3, 2018. The entire contents of the foregoing application are incorporated herein by reference in their entirety.

### BACKGROUND

### Technical Field

The present disclosure relates to systems and methods for tracking guideline concordance.

### Background Information

Guidelines are decision tools that provide evidence-based standards to help providers determine how to best treat a patient based on the patient's unique clinical factors, such as disease, stage, disease status and more. These guidelines are typically developed by authoritative professional societies and associations. A well-known provider of guidelines for oncology is the National Comprehensive Cancer Network (NCCN), but other institutions publish their own guidelines as well.

Health systems typically need to report clinical information to insurance companies and other payers to justify treatment decisions. The NCCN guidelines are a widely used source to determine what clinical data points are required to justify a given treatment decision. Physicians do not currently have a tool to efficiently capture clinical information associated with guideline concordance. For example, to obtain guideline-concordant treatment recommendations, physicians must navigate out of a typical workflow to review numerous pdf documents.

Having physicians capture and enter recommendation and guideline data points a is unduly expensive and time consuming, as there is no tool to seamlessly capture these data points in the physician workflow. Thus, without this data, it is often difficult to measure whether or not providers are following these guidelines.

In some instances, variation in care, e.g., the administration of non-concordant treatments, is associated with inferior patient outcomes. To improve patient outcomes, it is important to understand the relationship between guideline concordance and patient outcomes. However, the requires retrospective analysis of guideline concordance data or the use of a workflow-heavy clinical pathways tool that recommends a single treatment by taking existing guidelines, toxicity, and cost into consideration.

Accordingly, there is a need for a point of care solution that enables better guideline concordance reporting with minimal disruption to physician workflow and a better user experience around treatment selection through decision support. Such a point of care solution would facilitate data collection, thereby enabling retrospective analysis, and would support physician decision-making around treatment recommendations.

### SUMMARY

Embodiments consistent with the present disclosure include systems and methods for providing guideline concordance. In an embodiment, system for providing guideline concordance may comprise at least one processing device. The at least one processing device may be programmed to: receive, via a graphical user interface of a user device, a search term associated with a drug; access a structured database to identify, based on the search term, a description of at least one regimen that includes the search term; display, via the graphical user interface, a selectable identifier of the at least one regimen; receive, via the graphical user interface, a selection of a regimen, wherein the regimen is associated with the drug; generate, based on the structured database, one or more indications that are concordant for the regimen; receive, via the graphical user interface, a selection of a concordant indication; and store, in an electronic health record database, a patient record with information identifying the selected regimen and the selected indication.

In an embodiment, a method for providing guideline concordance may comprise receiving, via a graphical user interface of a user device, a search term associated with a drug; accessing a structured database to identify, based on the search term, a description of at least one regimen that includes the search term; displaying, via the graphical user interface, a selectable identifier of the at least one regimen; receiving, via the graphical user interface, a selection of a regimen, wherein the regimen is associated with the drug; generating, based on the structured database, one or more indications that are concordant for the regimen; receiving, via the graphical user interface, a selection of a concordant indication; and storing, in an electronic health record database, a patient record with information identifying the selected regimen and the selected indication.

Consistent with other disclosed embodiments, non-transitory computer readable storage media may store program instructions, which are executed by at least one processing device and perform any of the methods described herein.

### BRIEF DESCRIPTION OF THE DRAWINGS

The accompanying drawings, which are incorporated in and constitute part of this specification, and together with the description, illustrate and serve to explain the principles of various exemplary embodiments. In the drawings:

FIG. 1 is a block diagram illustrating an exemplary system environment for implementing embodiments consistent with the present disclosure.

FIG. 2 is a block diagram illustrating an exemplary process for implementing embodiments consistent with the present disclosure.

FIG. 3 is an illustration of an exemplary graphical user interface (GUI) consistent with the present disclosure.

FIG. 4A-4D are illustrations of exemplary GUIs consistent with the present disclosure.

FIG. 5 is an illustration of an exemplary GUI consistent with the present disclosure.

FIG. 6 is a flowchart illustrating an exemplary process for providing guideline concordance consistent with the present disclosure.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Embodiments herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, nonvolatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

Embodiments of the present disclosure provide systems and methods for providing guideline concordance. A user of the disclosed systems and methods may encompass any individual who may wish to access a patient's clinical experience and/or analyze patient data. Thus, throughout this disclosure, references to a “user” of the disclosed systems and methods may encompass any individual, such as a physician, a quality assurance department at a health care institution, and/or the patient. While reference is made to tumors or cancer therapies throughout this disclosure, these are provided as an example only, and it is understood that the disclosed systems and methods may apply to various other diseases and/or treatments.

Disclosed embodiments describe a point of care solution that enables better guideline concordance reporting with minimal disruption to physician workflow and better user experience around treatment selection through decision support. For example, the user interface may be the means through which a physician can report guideline concordance without having to use a pathways tool, thereby improving efficiency and providing a seamless process. Pathway tools are often inefficient and require the user to navigate through one or more pdf documents outside the workflow. Additionally, in some embodiments, the user interface may improve the overall treatment selection by providing clinical decision support. Disclosed embodiments may provide an application that seamlessly captures patient data points, which may, in turn, accelerate reimbursement approval, enhance negotiating strength, and reduce operational costs for cancer centers.

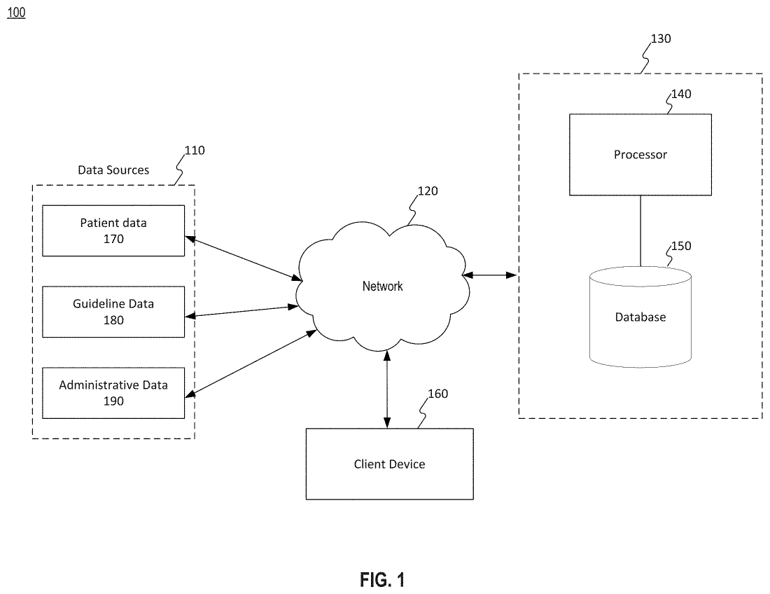

FIG. 1 illustrates an exemplary system environment 100 for implementing embodiments consistent with the present disclosure, described in detail below. As shown in FIG. 1, system environment 100 includes several components. It will be appreciated from this disclosure that the number and arrangement of these components is exemplary and provided for purposes of illustration. Other arrangements and numbers of components may be used without departing from the teachings and embodiments of the present disclosure.

As shown in FIG. 1, exemplary system environment 100 includes a system 130. System 130 may include one or more server systems, databases, and/or computing systems configured to receive information from entities over a network, process the information, store the information, and display/transmit the information to other entities over the network. Thus, in some embodiments, the network may facilitate cloud sharing, storage, and/or computing. In one embodiment, system 130 may include a processor 140 and one or more databases 150, which are illustrated in a region bounded by a dashed line representing system 130 in FIG. 1. Processor 140 may comprise at least one processing device, such as one or more generic processors, e.g., a central processing unit (CPU), a graphics processing unit (GPU), or the like and/or one or more specialized processors, e.g., an application-specific integrated circuit (ASIC), a field-programmable gate array (FPGA), or the like.

In one embodiment, system 130 may transmit and/or receive data to/from various other components, such as one or more data sources 110 and client device 160. More specifically, system 130 may be configured to receive and store the data transmitted over a network 120 (e.g., the Internet, an Intranet, WAN, LAN, cellular network, Bluetooth, etc.) from various data sources, including data sources 110, process the received data, and transmit data and results based on the processing to client device 160.

The various components of system environment 100 may include an assembly of hardware, software, and/or firmware, including a memory, a central processing unit (CPU), and/or a user interface. Memory may include any type of RAM or ROM embodied in a physical storage medium, such as magnetic storage including floppy disk, hard disk, or magnetic tape; semiconductor storage such as solid-state disk (SSD) or flash memory; optical disc storage; or magneto-optical disc storage. A CPU may include one or more processors for processing data according to a set of programmable instructions or software stored in the memory. The functions of each processor may be provided by a single dedicated processor or by a plurality of processors. Moreover, processors may include, without limitation, digital signal processor (DSP) hardware, or any other hardware capable of executing software. An optional user interface may include any type or combination of input/output devices, such as a display monitor, keyboard, and/or mouse.

Data transmitted and/or exchanged within system environment 100 may occur over a data interface. As used herein, a data interface may include any boundary across which two or more components of system environment 100 exchange data. For example, environment 100 may exchange data between software, hardware, databases, devices, humans, or any combination of the foregoing. Furthermore, it will be appreciated that any suitable configuration of software, processors, data storage devices, and networks may be selected to implement the components of system environment 100 and features of related embodiments.

As described further below, system 130 may be configured to receive patient data 170, guideline data 180, and/or administrative data 190, e.g., data indicating preferred treatments or available trials, from data sources 110 or other sources in network 120. Data sources 110 may include a variety of sources of medical, guideline, and administrative data. For example, data sources 110 may include medical care providers of the patient, such as physicians, nurses, specialists, consultants, hospitals, clinics, and the like. Data sources 110 may also include laboratories such as radiology or other imaging labs, hematology labs, pathology labs, etc. Data sources 110 may also include any other sources of patient, insurance, or guideline data.

In patient data 170, each patient may be represented by one or more records generated by one or more health care professionals or by the patient. For example, a doctor associated with the patient, a nurse associated with the patient, a physical therapist associated with the patient, or the like, may each generate a medical record for the patient. In some embodiments, one or more records may be collated and/or stored in the same database. In other embodiments, one or more records may be distributed across a plurality of databases. In some embodiments, the records may be stored and/or provided a plurality of electronic data representations. For example, the patient records may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process. Patient data 170 may be stored, for example, as electronic health records (EHRs) in an electronic health record database. In some embodiments, a patient EHR may include, for example, identification information (e.g., name, address, date of birth, etc.), medical history information (e.g., treatment dates, surgical history, prescribed medicines, family medical history, etc.), provider information (e.g., primary insurance provider, copay amount, secondary insurance provider), and/or contact information (e.g., emergency contact information, primary care provider contact information, etc.).

In some embodiments, guideline data 180 may be received from a guideline publisher, e.g., NCCN. Guideline data 180 may include a guideline compendium storing non-structured data. For example, a compendium may store non-structured decision tree data in a tabular format. In some embodiments, guideline data 180 may include one or more decision trees defining guidelines and/or pathways associating clinical attributes and/or indications with one or more treatment regimens. In some embodiments, one or more guideline documents may be collated and/or stored in the same database. In other embodiments, one or more guideline documents may be distributed across a plurality of databases. In some embodiments, the guidelines may be stored and/or provided a plurality of electronic data representations. For example, the guidelines may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process.

Administrative data 190 may be data received from an insurance provider or medical personnel, e.g., hospital administrator. The administrator data may indicate one or more preferred pathways or may indicate treatments covered by a patient's insurance. In some embodiments, insurance data for a particular patient may be provided as patient data 170. Administrative data 190 may be received from an administrative system, e.g., a hospital system or hospital database and may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process.

System 130 may further communicate with one or more client devices 160 over network 120. For example, system 130 may provide a list of regimens in response to a search performed by a physician based on analysis of data from data sources 110 to client device 160. Client device 160 may include any entity or device capable of receiving or transmitting data over network 120. For example, client device 160 may include a computing device, such as a server or a desktop or laptop computer. Client device 160 may also include other devices, such as a mobile device, a tablet, a wearable device (i.e., smart watches, implantable devices, fitness trackers, etc.), a virtual machine, an IoT device, or other various technologies. In some embodiments, client device 160 may transmit queries for information about a patient and/or about a treatment over network 120 to system 130, such as a query for a particular treatment, patient medical records, or various other information about a patient.

Guideline data 180 may be received from data sources 110 and processed by system 130, as described above. The guidelines received from data sources 110 (or elsewhere) may include one or more decision trees. The decision trees may guide a provider through the process of selecting a treatment based on a patient's clinical attributes. Traditionally, a provider navigates through a number of decision trees stored as pdf documents to identify one or more guideline concordant treatments for the patient based on the patient's clinical attributes. This method may disrupt the practitioner workflow, resulting in inefficiencies and inconsistent tracking of guideline concordance. For example, using guideline decision trees may require a practitioner to navigate numerous pdfs and make a number of decisions before arriving at a treatment recommendation. Further, this traditional approach lacks flexibility since the practitioner is required to follow an ordered sequence of decisions.

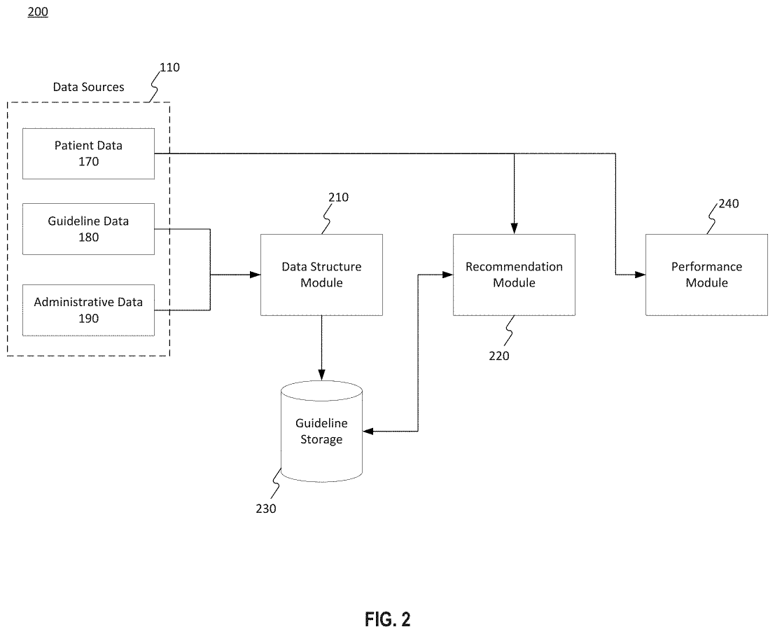

FIG. 2 illustrates system 200, which is an exemplary embodiment of system 130 for implementing embodiments consistent with the present disclosure. System 200 may be implemented as part of system 130 (FIG. 1). For example, system 200 be a component or process of processor 140. In some embodiments, system 130 may parse one or more decision trees received from data sources 110 to identify relationships between clinical attributes and treatments. System 130 may also identify negative relationships between clinical attributes and treatments, thereby obviating a practitioner's need to evaluate a decision point that does not apply to a particular patient.

Data structure module 210 may be configured to receive guideline data 180 and administrative data 190 from data sources 110, e.g., via a data interface. Guideline data 180 and administrative data 190 may be received from one or more local or remote databases. In some embodiments, data structure module 210 may be configured to receive structured input associated with non-structured compendium records. For example, a compendium may store guidelines by drug name. For each drug, the compendium may include free-form block text describing decisions required to reach a treatment recommendation. In some embodiments, a structured data

Data structure module 210 may receive input indicative of pathways and/or guidelines defined in the compendium and store these pathways and/or guidelines in a structured database, e.g., guideline storage 230. For example, a data structure may be generated for each drug, pathway, and/or guideline stored in the compendium. The data structure may be configured to relate a treatment regimen to one or more guideline concordant clinical attributes. In some embodiments, the treatment regimen may be related to one or more indications, where an indication includes one or more patient attributes. For example, a treatment regimen may be guideline concordant if the regimen is indicated for the patient receiving the regimen, i.e., if the patient presents the patient attributes of an indication associated with the treatment regimen.

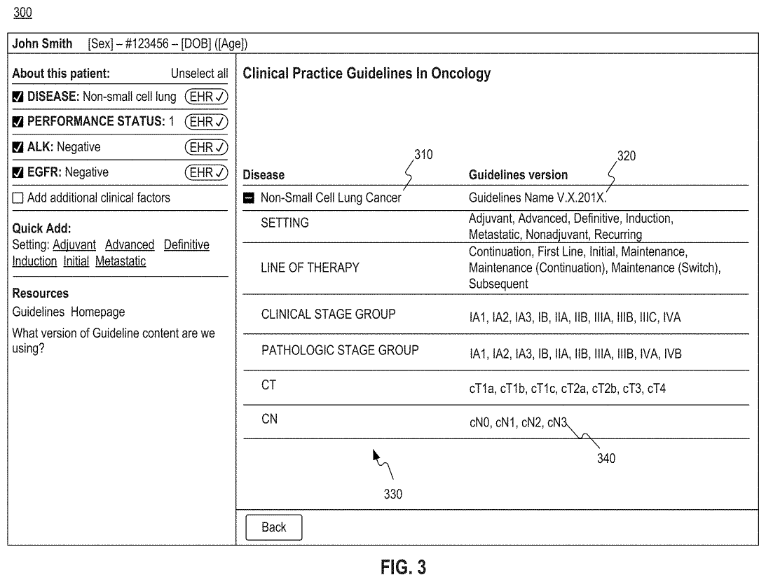

Recommendation module 220 may be configured to receive input from data sources 110 and from client device 160. For example, a user may input data via a graphical user interface (GUI) displayed by client device 160. Exemplary GUIs displayed by client device 160 are described in further detail below with reference to FIGS. 3, 4A-4D, and 5.

Recommendation module 220 may receive input of a search term or of one or more clinical attributes associated with a patient. In some embodiments, the search term may be a drug name, an indication, or a clinical attribute. Recommendation module 220 may query data storage 230 to identify one or more records containing the search term. In some embodiments, the recommendation module 220 may receive, associated with the search, patient data 170. The patient data 170 may be associated, for example, with a particular patient and may include one or more clinical attributes of the patient. Recommendation module 220 may generate a query of the data structure stored by data storage 230 in which the search results are filtered by indication and/or clinical attribute.

In other embodiments, recommendation module 220 may search or filter guideline storage 230 to identify one or more regimens that are concordant for a patient, based on the patient's clinical attributes. For example, recommendation module 220 may filter guideline storage 230 by one or more clinical attributes. In some embodiments, recommendation module 220 may be further configured to filter search results for a drug name by one or more clinical attributes.

Recommendation module 220 may execute the generated query and return a list of one or more regimens associated with the search term and/or clinical attributes. In some embodiments, the list may be ranked by relevance. For example, a relevance score for each regimen may be determined based on a comparison of the search term to a drug name, indication, or clinical attribute of the regimen. Thus, the most relevant regimens or results may be displayed at the top of the list. In other embodiments, search results may be listed with treatments preferred by a hospital or insurance provider listed first.

Recommendation module 220 may be configured to receive input, via client device 160, indicative of a selection of a regimen returned by the search. Responsive to the selection, recommendation module 220 may generate a GUI configured to display one or more concordant indications associated with the selected regimen. Each concordant indication may include a set of clinical attributes that a patient should have in order for the administered regimen to be guideline concordant.

Responsive to a selection of a concordant indication, in some embodiments, recommendation module 220 may prompt the user to enter additional detail associated with the regimen. For example, the user may be prompted to enter a dosage, frequency, route, start date of the regimen, end date of the regimen, and the like. In some embodiments, the user may select a regimen for a patient who does not present clinical attributes associated with a concordant information. If the user selects a non-concordant regimen, the user may be prompted to enter a reason for selecting a non-concordant regimen.

In some embodiments, recommendation module 220 may receive the regimen selection, indication selection, and regimen details and update an EHR associated with the patient with the received information. The EHR may be stored in an EHR database, e.g., database 150, or a remote database. In some embodiments, some or all of the received data may be stored in a reporting database.

In some embodiments, a performance module 240 may receive, from an EHR database, e.g., database 150, a reporting database, and/or an EHR database, aggregated patient data indicating patient regimens and outcomes. For example, performance module 240 may be configured to generate one or more reports comparing outcomes of concordant versus non-concordant patients. Thus, the system 200 may enable a hospital or other patient service provider to track concordance and/or identify trends in patient outcomes thereby leveraging the data generated via recommendation module 220.

FIGS. 3, 4A-4D, and 5 illustrate exemplary graphical user interfaces (GUIs) consistent with disclosed embodiments. The GUIs of FIGS. 3, 4A-4D, and 5 may be displayed to a user via client device 160, which may be capable of receiving user input via keyboard, microphone, or other I/O device.

As shown in FIG. 3, the user may view the underlying data model, e.g., guideline data stored in guideline database 230, via GUI 300. For example, GUI 300 may display a disease 310 associated with the patient, which may be determined based on the patient's EHR. GUI 300 may also display the guideline version 320. The guidelines indicated via GUI 300 may be e.g., guideline data 180 and may be used by processor 140 to generate outputs including suggested regimens and concordant regimens based on a patient's clinical attributes.

In some embodiments, the user may view, via GUI 300, a list of guideline parameters 330. Guideline parameters 330 may be, for example, column names and allowed values associated with guideline database 230. For example, each record of a regimen in database 230 may be stored with each of the parameters 330 having a value from each listing 340 of available values for the parameter. GUI 300 may further assist the user by indicating fields, e.g., parameters 330, by which the user may filter database 230 to identify regimens.

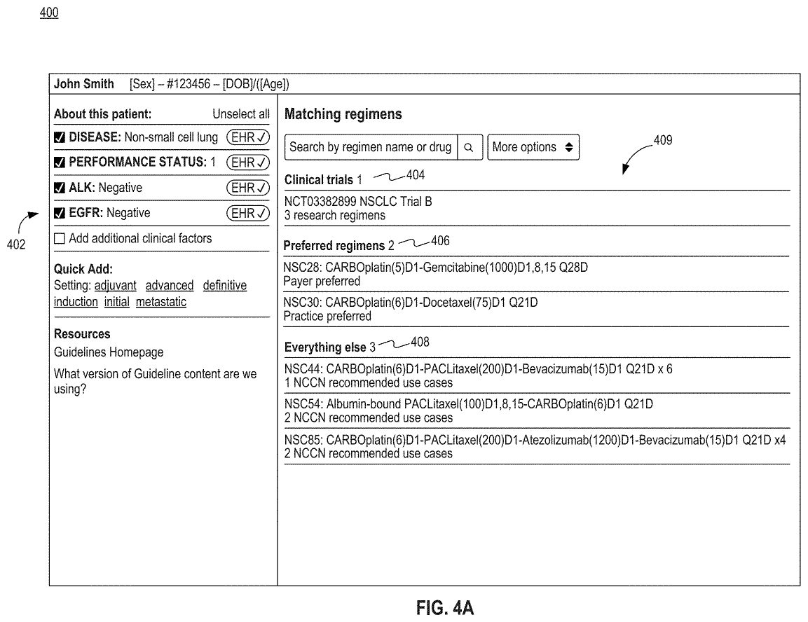

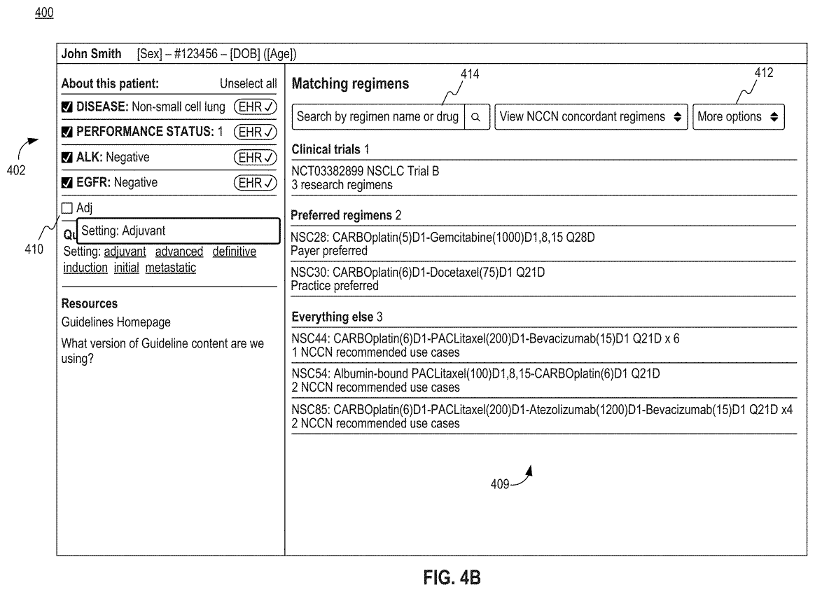

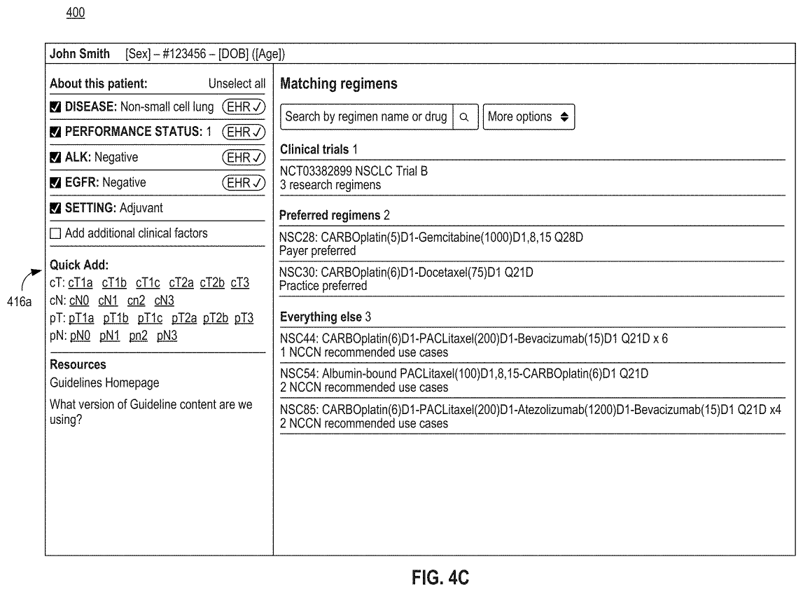

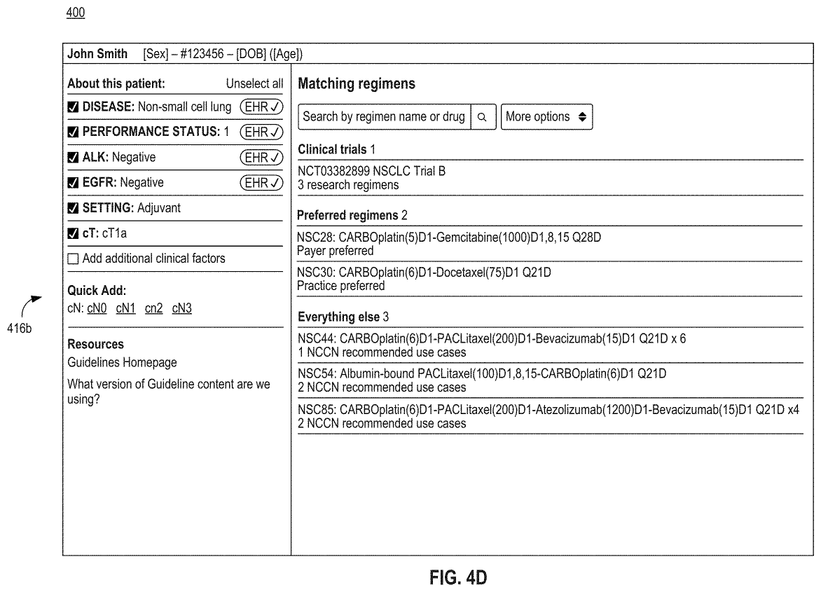

In some embodiments, a user may select a regimen to add to a patient's EHR as described with reference to FIGS. 4A-4D.

For example, as shown in FIG. 4A, a GUI 400 may display one or more regimens determined by recommendation module 220. For example, recommendation module 220 may receive patient data 170 as well as data from guideline storage 230. Recommendation module 220 may generate a list 402 of one or more patient attributes based on patient data 170. For example, each clinical attribute of list 402 may be pulled from structured data in a patient's EHR. Recommendation module may then query guideline storage 230 using these attributes to filter guideline concordant regimens for the patient.

GUI 400 may be configured to display a list of search results 409. The search results may be generated based on a query of one or more regimen databases as described above with reference to recommendation module 220. The regimen database may be, e.g., database 150 of system 100, or a remote database, e.g., a database associated with guideline data 180. For example, recommendation module 220 may generate a query to retrieve concordant regimens from guideline database 230 by filtering database records based on the values of filters 702.

In some embodiments, data structure module 210 may receive administrative data 190 indicative of practice or payer preferred treatments, and/or clinical trial regimens. For example, data structure module 210 may be configured to receive administrative data 190, which may include information associated with one or more clinical trials. Data structure module 210 may be configured to generate structured database records for each clinical trial, such that the available clinical trials are searchable in data storage 230. Similarly, in some embodiments, the system may receive, from recommendation module 220, administrative data 190 indicative of preferred treatments, e.g., treatments preferred by an insurance company, or of potential clinical trials. This data may also be displayed to the user via GUI 400 as part of the list of concordant regimens. Thus, GUI 400 may be used to indicate to the user, e.g., the physician, preferred treatments or trials available for a patient having a particular set of clinical attributes. For example, GUI 400 may be configured to display, in the list of results 409, a list of concordant clinical trials 404, preferred regimens 406, and/or other concordant regimens 408 based on administrative data 190.

In some embodiments, the system may be further configured to retrieve trial regimen data from a database, e.g., data source 110. The system may generate GUI 400 to display one or more trial details in addition to the trial regimen. Trial details may indicate, for example, inclusion characteristics and exclusion characteristics to aid the user in determining whether the patient is eligible for the trial.

FIG. 4B illustrates another view of GUI 400 in which a user may add to the list of filters 402. For example, the user may enter free text in input field 410. In some embodiments, GUI 400 may automatically suggest a filter or filter value based on the text input and based on data stored by guideline database 230. As an example, recommendation module 220 may be configured to generate suggested filters based on parameters stored in guideline database 230, e.g., as shown via GUI 300 described with reference to FIG. 3.

In some embodiments, via GUI 400, a user may select from a drop-down menu of “More options” 412. For example, the user may select to further filter the results by guideline concordant regimens, or may select to view unavailable guideline templates. Thus, the user may view regimens matching a search term input via search field 414, rather than the list of attributes 402 pulled from the patient's EHR. In this embodiment, recommendation module 220 may query guideline database 230 using the search term received via search field 414 and return one or more regimens associated with the search term, e.g., where the regimen record in guideline database 230 matches or contains the search term.

For example, if a user selects to view unavailable regimens, the user may select a regimen displayed via GUI 400 even if the patient does not have clinical attributes of any concordant indication. In this case, the user may select a reason for administering a non-concordant treatment regimen. The reason may be selected from a drop-down menu. In other embodiments, a GUI may include a text field configured to receive free form input. In some embodiments, the user must select a reason for administering a non-concordant treatment regimen prior to continuing the workflow.

In some embodiments, once the user selects a regimen or trial regimen from list 409, e.g., via a touchscreen or I/O device of client device 160, the system may generate a GUI configured to display a list of concordant indications associated with the selected regimen. Each concordant indication may include a set of clinical attributes that should be present in the patient for the regimen to be concordant and may list and/or otherwise indicate attributes of the patient based on structured data pulled from the patient's EHR. For example, a concordant indication may be associated with the clinical attributes of: a cancer type, e.g., non-small cell lung cancer (NSCLC), tumor, node, and metastasis (TNM) staging information, e.g., T2aN0M0 or T2bN0M0, therapy type, e.g., adjuvant, and a residual tumor classification, e.g., R0. Other clinical attributes may be associated with an indication.

In some embodiments, GUI 400 may be configured to receive a selection of a regimen from the list of search results 409, and, responsive to the selection, the GUI may display a pathway associated with the selected regimen. In other embodiments, a GUI may be configured to display an indication of payer or provider preferred regimens or clinical trial regimens associated with the received search term.

As shown in FIG. 4C, in some embodiments, GUI 400 may be configured to display a list 416a of “Quick Add” attributes. The list 416a may be generated dynamically based on the patient's clinical attributes and on the guidelines. For example, the additional “Quick Add” attributes may be determined based on criteria for pathways matching the patient's clinical attributes. The user may select from the “Quick Add” list 416a to further narrow the search criteria applied to guideline database 230. Other exemplary filters, not shown, may include bio-markers, line of therapy, pathologic stage group, and the like. Available filters may be based on, for example, the parameters of the selected guidelines, as shown in FIG. 3.

FIG. 4D displays another view of GUI 400 in which a user has selected a cT value of cT1a from the Quick Add list 416a. Recommendation module 220 may then dynamically retrieve available attributes by which to further filter available regimens and display those attributes as Quick Add list 416b. For example, by selecting cT1a, the user may then only select further filters from the list of cN values, for which concordant regimens are available. In another embodiment, the filters displayed in the Quick Add lists 416a and 416b may be determined based on whether the presence of a certain attribute eliminates the possibility of a patient possessing another attribute.

In another embodiment, GUI 400 may be configured to generate, based on data structures generated by data structure module 210, a decision tree associated with the search term input via field 414, or the filter values 402 pulled from the patient's EHR. In some embodiments, the displayed decision tree may be dynamic, such that a user may select from one or more links to view pathways of the generated decision tree. In some embodiments, the displayed decision tree may be generated, in part, based on patient data. For example, the displayed pathways may be selected based on data retrieved from the patient's EHR. If the patient's medical history indicates the patient's NSCLC is in a pathologic stage pT2a, NO or pT2b, NO, GUI 640 may display the associated decision tree.

In some embodiments, after a user selects a regimen, the system may generate a GUI configured to receive additional detail regarding the selected regimen. For example, the user may input information including, for example, a treatment start date, a treatment end date, a line of treatment, a treatment goal, a treatment plan provider, and/or a treatment department, respectively. In some embodiments, some or all of the additional information may be optional. Additional details regarding the patient may be retrieved from guideline storage 230 or from another database storing regimen information. In some embodiments, additional information, e.g., dosage information or demographics may be retrieved from the patient's EHR.

After inputting and reviewing the information input, a user may add the treatment plan to the patient's EHR. The system may update and/or save the patient EHR with the added treatment plan to an EHR database. In some embodiments, all or part of the information may be saved to a reporting database. After accepting the treatment plan, a GUI of system 130 may display a summary of the received treatment plan data. For example, treatment plan information may be accessed at any time by the user through the patient's EHR.

As previously described, the system may save treatment plan data received via GUI 400 to a reporting database. The system may be used, for example, by a hospital administrator to generate one or more reports associated with aggregate guideline concordance data.

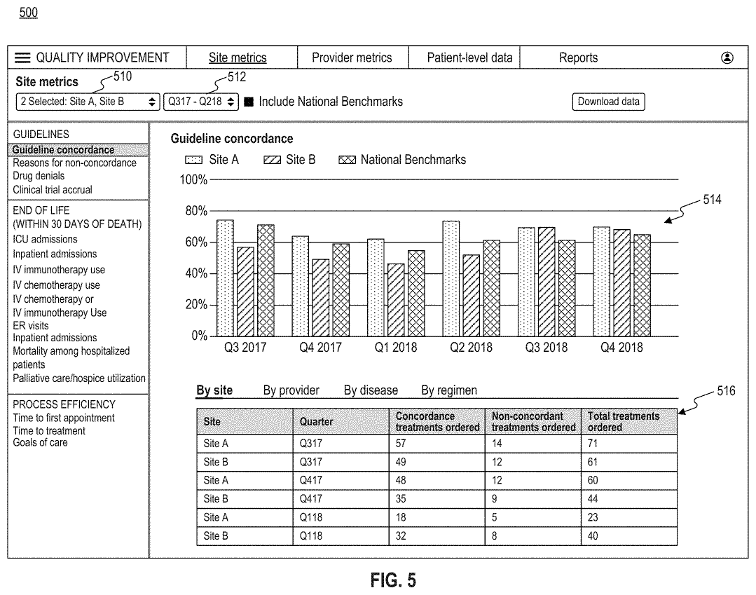

FIG. 5 illustrates a GUI 500 displaying an exemplary guideline concordance report. Via GUI 500 a user may access an EHR database and/or reporting database to analyze aggregate data. For example, GUI 500 may be configured to receive one or more parameters, e.g., a site 510 and a date range 512. In other embodiments, GUI 500 may be configured to receive any number of parameters, e.g., a date, a regimen, a medical provider, etc. In other embodiments, GUI 500 may be configured to receive a structured database query.

GUI 500 may include a graph view 514 and a tabular view 516. Performance module 240 may be configured to receive, via GUI 500, the one or more parameters and generate a query of the reporting database and/or EHR database. Based on the query results, performance module 240 may generate a visual representation, e.g., graph 514, of the returned data. Performance module 240 may also generate a table 516, which may be filtered by site, provider, disease, or regimen. In yet another embodiment, performance module 240 may query one or more databases storing national standard data and display, via GUI 500, benchmark data.

GUI 500 may enable administrators and providers to view summaries and trends of concordance data, which may be useful in guiding treatment decisions or identifying preferred treatments. GUI 500 may also be configured to display data associated with administered non-concordant regimens and/or patient outcomes. Thus, a provider or administrator may be better able to leverage concordance data to improve patient outcomes and patient care.

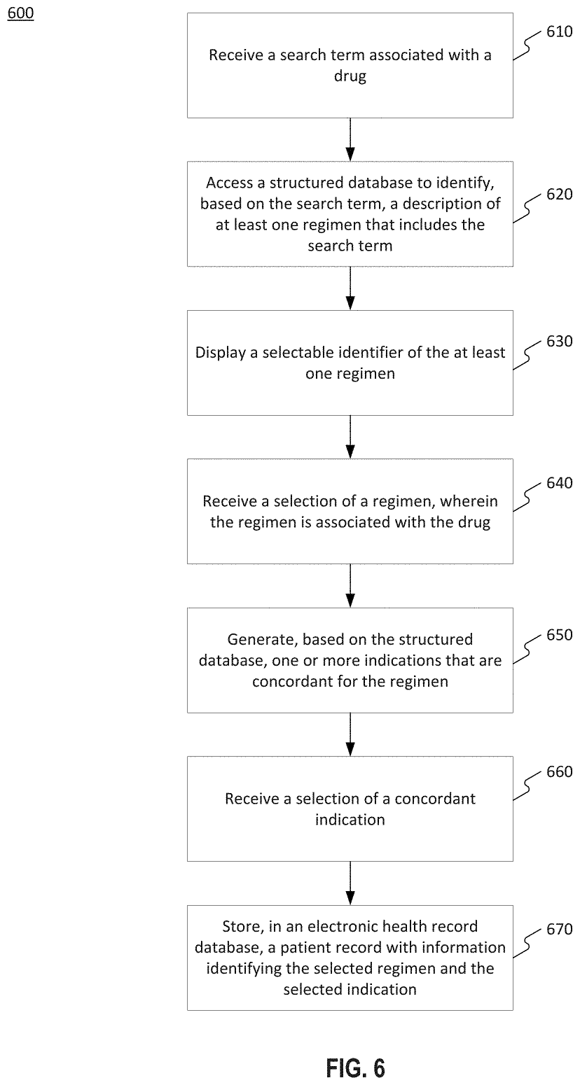

FIG. 6 illustrates an exemplary process 600 for providing guideline concordance. Process 600 may be implemented, for example, a processor 140 of system 100, shown in FIG. 1.

At step 610, the system may receive a search term associated with a drug. For example, a user may input a search term via GUI 400, or system 130 may pull patient information from a patient's EHR and perform a search using the patient's clinical attributes. In other embodiments, the user may search by indication and/or clinical attribute to identify regimens associated with the input indication and/or clinical attribute.

At step 620, processor 140 may access a structured database to identify, based on the search term, a description of at least one regimen that includes the search term. For example, based on the search term, processor 140 may query guideline storage 230 to identify at least one regimen containing or matching the search term. In some embodiments, the query may identify at least one regimen containing a partial match of the search term. In some embodiments, processor 140 may identify a regimen based on whether the regimen name, description, or other associated information contain at least a partial match of the search term.

As previously described, the structured database may be generated by data structure module 210 and may be based on a guideline compendium containing non-structured data. In some embodiments, the guideline compendium may include a number of decision trees. Data structure module 210 may generate structured guideline records automatically or may be configured to receive input from an administrator. Data structure module 210 may be configured to identify and store relationships between clinical attributes and regimens, thereby enabling a physician to identify appropriate treatment regimens, without needing to navigate through an ordered list of decisions.

At step 630, processor 140 may display, via a graphical user interface, a selectable identifier of the at least one regimen. For example, processor 140 may generate a GUI, as shown in FIGS. 4A-4D. Processor 140 may transmit the search results to client device 160, thereby causing client device 160 to display the GUI. In embodiments in which the search term is an indication or clinical attribute, the GUI may display a list of regimens that are associated with the indication and/or clinical attribute.

As previously described, processor 140 may determine a relevance score associated with each record based on, for example, a number of times the search term appears in the record, or whether one or more patient attributes match the clinical attributes associated with the record. The GUI may be configured to display a ranked list of search results, where the most relevant results are listed first. In another embodiment, the GUI may display a predetermined number of results, e.g., the top 20 results.

At step 640, processor 140 may receive a selection of a regimen. For example, the user may click on or hover over a regimen on a display of client device 160. The selection may be transmitted to processing device 140 via network 120. In some embodiments, processor 140 may be a processor of client device 160. The selection may be received, for example, as a result of the user clicking on the regimen with a cursor or hovering over the regimen. In some embodiments, the GUI may include one or more of a radio button, a link, or a checkbox configured to receive a selection from the user.

At step 650, responsive to the user's selection of a regimen at step 640, processor 140 may generate, based on the structured database, one or more indications that are concordant for the regimen. For example, recommendation module 220 may generate a query of data storage 230 configured to retrieve data associated with the selected regimen. Data associated with the regimen may include one or more concordant indications and/or clinical attributes, where an indication may be a set of one or more clinical attributes.

At step 660, processor 140 may receive a selection of a concordant indication. For example, in some embodiments, the user may select a concordant indication with which the patient shares clinical attributes. As previously described, in some embodiments, the patient may not match any concordant indications. In this case, the user may select to proceed with the non-concordant regimen. In some embodiments, the system may require a user to input a reason for selecting a non-concordant treatment for the patient. The input may be received, for example, as free form text, or may be a prepopulated reason selected from a drop-down menu.

In other embodiments, the retrieved search results may be filtered based on the patient's clinical attributes. In such embodiments, the displayed list of regimens may all be guideline concordant. In some embodiments, e.g., via GUI 400, a user may select whether to view only concordant regimens or view non-concordant regimens.

At step 670, processor 140 may store, in an electronic health record database, a patient record with information identifying the selected regimen and the selected indication. For example, processor 140 may update a patient EHR in an EHR database. In some embodiments, the input received in process 600 may also be stored in a reporting database.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, 4K Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, Python, R, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A system for providing guideline concordance, the system comprising:
at least one processing device programmed to:
receive, via a graphical user interface of a user device, a search term associated with a drug;
access a structured database to identify, based on the search term, a description of at least one regimen that includes the search term;
display, via the graphical user interface, a selectable identifier of the at least one regimen;
receive, via the graphical user interface, a selection of a regimen, wherein the regimen is associated with the drug;
generate, based on the structured database, one or more indications that are concordant for the regimen;
receive, via the graphical user interface, a selection of a concordant indication; and
store, in an electronic health record database, a patient record with information identifying the selected regimen and the selected indication.

2. The system of claim 1, wherein the structured database is based on a guideline compendium.

3. The system of claim 2, wherein the structured database comprises non-structured data of the guideline compendium.

4. The system of claim 2, wherein the guideline compendium comprises a plurality of decision trees associating indications with treatments for a plurality of drugs.

5. The system of claim 1, wherein each of the one or more indications comprises at least one patient attribute.

6. The system of claim 1, wherein the electronic health record database comprises aggregated regimen data from a plurality of input sources.

7. The system of claim 1, wherein the at least one processing device is further programmed to:
receive, via the graphical user interface, a selection of a non-concordant indication and non-structured text describing the reason for selecting a non-concordant regimen.

8. The system of claim 7, wherein the at least one processing device is further configured to:
update a log storing non-concordant regimen information.

9. The system of claim 1, wherein the at least one processing device is further configured to:
receive one or more patient characteristics; and
access the structured database to identify, based on the search term and the one or more patient characteristics, a description of at least one regimen that includes the search term and is associated with the one or more patient characteristics.

10. A method for providing guideline concordance, the method comprising:
receiving, via a graphical user interface of a user device, a search term associated with a drug;
accessing a structured database to identify, based on the search term, a description of at least one regimen that includes the search term;
displaying, via the graphical user interface, a selectable identifier of the at least one regimen;
receiving, via the graphical user interface, a selection of a regimen, wherein the regimen is associated with the drug;
generating, based on the structured database, one or more indications that are concordant for the regimen;
receiving, via the graphical user interface, a selection of a concordant indication; and
storing, in an electronic health record database, a patient record with information identifying the selected regimen and the selected indication.

11. The method of claim 10, wherein the structured database is based on a guideline compendium.

12. The method of claim 11, wherein the structured database comprises non-structured data of the guideline compendium.

13. The method of claim 11, wherein the guideline compendium comprises a plurality of decision trees associating indications with treatments for a plurality of drugs.

14. The method of claim 10, wherein each of the one or more indications comprises at least one patient attribute.

15. The method of claim 10, wherein the electronic health record database comprises aggregated regimen data from a plurality of input sources.

16. The method of claim 10, wherein the method further comprises:
receiving, via the graphical user interface, a selection of a non-concordant indication and non-structured text describing the reason for selecting a non-concordant regimen.

17. The method of claim 16, wherein the method further comprises:
updating a log storing non-concordant regimen information.

18. The method of claim 10, wherein the method further comprises:
receiving one or more patient characteristics; and
accessing the structured database to identify, based on the search term and the one or more patient characteristics, a description of at least one regimen that includes the search term and is associated with the one or more patient characteristics.

19. A computer-readable medium storing instructions that when executed cause a processor to:
receive, via a graphical user interface of a user device, a search term associated with a drug;
access a structured database to identify, based on the search term, a description of at least one regimen that includes the search term;
display, via the graphical user interface, a selectable identifier of the at least one regimen;
receive, via the graphical user interface, a selection of a regimen, wherein the regimen is associated with the drug;
generate, based on the structured database, one or more indications that are concordant for the regimen;
receive, via the graphical user interface, a selection of a concordant indication; and
store, in an electronic health record database, a patient record with information identifying the selected regimen and the selected indication.
